(19) United States
(12) Patent Application Publication
(10) Pub. No.: US 2006/0204588 A1
(43) Pub. Date: Sep. 14, 2006

(54) Formulations of a nanoparticulate finasteride, dutasteride or tamsulosin hydrochloride, and mixtures thereof

(75) Inventors: Gary Liversidge, Westchester, PA (US); Scott Jenkins, Downingtown, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/372,227

(22) Filed: Mar. 10, 2006

Related U.S. Application Data
(60) Provisional application No. 60/660,229, filed on Mar. 10, 2005.

(51) Int. Cl. A61K 9/50; A61K 9/16

(52) U.S. Cl. 424/490; 977/906

(57) ABSTRACT

Described are nanoparticulate compositions of finasteride, dutasteride, tamsulosin hydrochloride, or a combination thereof. The formulations exhibit unexpectedly prolonged release and can be maintained in a depot for release to a patient for a period of up to six months.

### BACKGROUND OF THE INVENTION

1. Field of the Invention

The invention is directed to a nanoparticulate formulations of finasteride, dutasteride, or tamsulosin hydrochloride, or any combination thereof. The compositions of the invention, which surprisingly can be formulated into injectable depot dosage forms, are particularly useful in the treatment of benign prostatic hyperplasia. The invention also comprises methods of making and using such formulations.

2. Description of the Related Art

A. Background Regarding the Compounds of the Invention and Methods of Treatment

1. Finasteride

Finasteride is a synthetic androgen inhibitor used primarily in men for the treatment of benign prostatic hyperplasia and androgenetic alopecia (hairloss). Finasteride, a synthetic, 4-azasteroid compound, is a specific inhibitor of steroid Type II 5α-reductase, an intracellular enzyme that converts the androgen testosterone into 5α-dihydrotestosterone.

The compound is known chemically as (5alpha,17beta)-N-(1,1-dimethylethyl)-3-oxo-4-azaandrost-1-ene-17-carboxamide. Finasteride is insoluble in water and soluble in chloroform and alcohol. The empirical formula of finasteride is C₂₃H₃₆N₂O₂ and its molecular weight is 372.55. Finasteride has the following structure:

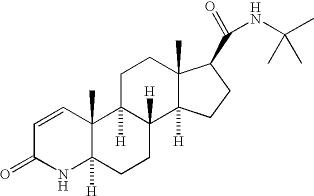

Finasteride is a white crystalline powder with a melting point near 250° C. It is freely soluble in chloroform and in lower alcohol solvents, but is practically insoluble in water. Finasteride is commercially available under the trade name PROSCAR®. PROSCAR® tablets (Merck & Co., Inc. (West Point, Pa.)) for oral administration are film-coated and contain 5 mg of finasteride and the following inactive ingredients: hydrous lactose, microcrystalline cellulose, pregelatinized starch, sodium starch glycolate, hydroxypropyl cellulose LF, hydroxypropylmethyl cellulose, titanium dioxide, magnesium stearate, talc, docusate sodium, FD&C Blue 2 aluminum lake and yellow iron oxide.

PROSCAR® is recommended for the treatment of symptomatic benign prostatic hyperplasia in men with an enlarged prostate to: improve symptoms; reduce the risk of acute urinary retention; reduce the risk of the need for surgery, including transurethral resection of the prostate and prostatectomy. Physician's Desk Reference 58^{th} Edition (Thompson PDR, Montvale, N.J., 2004) pp. 10, 325 and 2070-73.

2. Dutasteride

Dutasteride is a synthetic 4-azasteroid compound which is an antiandrogen which inhibits the conversion of testosterone into dihydrotestosterone. Clinical studies have found it to be more effective than finasteride in doing so, as it inhibits both isoforms of steroid 5-alpha reductase (5AR), an intracellular enzyme that converts testosterone to dihydrotestosterone (DHT). Dutasteride is indicated for the treatment of symptomatic BPH in men with an enlarged prostate to: improve symptoms, reduce the risk of acute urinary retention, and reduce the risk of the need for BPH-related surgery. Dutasteride is currently in trial phase for the treatment of alopecia (hairloss).

Dutasteride is known chemically as (5α, 17β)-(2,5 bis-(trifluoromethyl)phenyl)-3-oxo-4-azaandrost-1-ene-17-carboxamide. The empirical formula is C₂₇H₃₀F₆N₂O₂, representing a molecular weight of 528.5. The compound has the following structure:

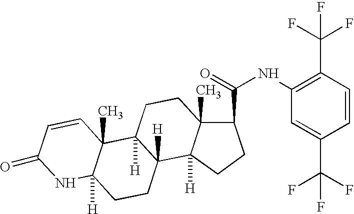

Dutasteride is a white to pale yellow powder with a melting point of 242° C. to 250° C. It is soluble in ethanol (44 mg/mL), methanol (64 mg/mL) and polyethylene glycol 400 (3 mg/mL), but it is insoluble in water.

Dutasteride is commercially available under the trade name AVODART®. AVODART® Soft Gelatin Capsules (GlaxoSmithKline (Research Triangle Park, N.C.)) for oral administration contain 0.5 mg of the active ingredient dutasteride in yellow capsules with red print. Each capsule contains 0.5 mg dutasteride dissolved in a mixture of mono-di-glycerides of caprylic/capric acid and butylated hydroxytoluene. The inactive excipients in the capsule shell are gelatin (from certified BSE-free bovine sources), glycerin, and ferric oxide (yellow). The soft gelatin capsules are printed with edible red ink.

AVODART® (dutasteride) is a synthetic 4-azasteroid compound that is a selective inhibitor of both the type 1 and type 2 isoforms of steroid 5α-reductase (5AR), an intracellular enzyme that converts testosterone to 5α-dihydrotestosterone. Physician's Desk Reference, 58^{th} Ed. (Thompson PDR, Montvale, N.J., 2004) pp. 316 and 1456-59.

3. Tamsulosin Hydrochloride

Tamsulosin hydrochloride is an antagonist of alpha_{1A} adrenoceptors in the prostate. This drug is used clinically as an oral medication to ameliorate the dysuria associated with prostatic hypertrophy.

Tamsulosin hydrochloride is known chemically as (−)-(R)-5-[2-[[2-(0-ethoxyphenoxy) ethyl]amino]propyl]-2-methoxybenzenesulfonamide, monohydrochloride. Tamsulosin hydrochloride occurs as white crystals that melt with decomposition at approximately 230° C. It is sparingly soluble in water and in methanol, slightly soluble in glacial acetic acid and in ethanol, and practically insoluble in ether. The compound has the following structure: 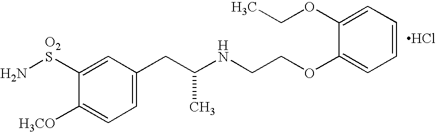
The empirical formula of tamsulosin hydrochloride is C₂₀H₂₈N₂O₅S.HCl. The molecular weight of tamsulosin hydrochloride is 444.98.

Tamsulosin hydrochloride is commercially available under the trade name FLOMAX®. FLOMAX® capsules (Boehringer Ingelheim (Ridgefield, Conn.)) for oral administration contain tamsulosin hydrochloride 0.4 mg, and the following inactive ingredients: methacrylic acid copolymer, microcrystalline cellulose, triacetin, polysorbate 80, sodium lauryl sulfate, calcium stearate, talc, FD&C blue No. 2, titanium dioxide, ferric oxide, gelatin, and trace amounts of shellac, industrial methylated spirit 74 OP, n-butyl, alcohol, isopropyl alcohol, propylene glycol, dimethylpolysiloxane, and black iron oxide E172.

Tamsulosin, an alpha₁ adrenoceptor blocking agent, exhibits selectivity for alpha₁ receptors in the human prostate. At least three discrete alpha₁-adrenoceptor subtypes have been identified: alpha_{1A}, alpha_{1B} and alpha_{1D}; their distribution differs between human organs and tissue. Approximately 70% of the alpha₁-receptors in the human prostate are of the alpha_{1A} subtype. Physician's Desk Reference, 58^{th} Edition (Thompson PDR, Montvale, N.J., 2004) pp. 4, 310 and 1006.

4. Treatment of Prostatic Hyperplasia

The prostate gland is located around the tube which empties urine from the bladder (urethra). As the prostate gland enlarges, usually after 50 years of age, it can obstruct or partially block the urine flow. This leads to symptoms which include dribbling of urine, narrow stream, problems starting urine flow, interruption while urinating, and a feeling of incomplete emptying. Other symptoms include wetting and staining of clothes, urinary burning, and urgency.

Prostate gland enlargement (Benign Prostatic Hyperplasia or BPH), is directly dependent on DHT (a hormone converted from the male hormone testosterone). Finasteride inhibits the enzyme necessary for the conversion of testosterone to DHT in the prostate. Therefore, administration of finasteride lowers blood and tissue DHT levels and helps reduce the size of the prostate gland.

The symptoms associated with benign prostatic hyperplasia are related to bladder outlet obstruction, which is comprised of two underlying components: static and dynamic. The static component is related to an increase in prostate size caused, in part, by a proliferation of smooth muscle cells in the prostatic stroma. However, the severity of benign prostatic hyperplasia symptoms and the degree of urethral obstruction do not correlate well with the size of the prostate. The dynamic component is a function of an increase in smooth muscle tone in the prostate and bladder neck leading to constriction of the bladder outlet. Smooth muscle tone is mediated by the sympathetic nervous stimulation of alpha₁ adrenoceptors, which are abundant in the prostate, prostatic capsule, prostatic urethra, and bladder neck. Blockade of these adrenoceptors can cause smooth muscles in the bladder neck and prostate to relax, resulting in an improvement in urine flow rate and a reduction in symptoms of benign prostatic hyperplasia.

Treatment of benign prostatic hyperplasia is generally required over the remaining life of a patient. Current pharmaceutical compositions used in such treatment which are typically in the form of tablets or capsules taken daily, are inconvenient as they require ongoing patient compliance. The administration of such dosages may be forgotten, which lessens the efficacy of the treatment. Alternative dosage forms of drugs useful in treating BPH are therefore desirable.

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto or associated with the surface thereof a non-crosslinked surface stabilizer. The '684 patent does not describe nanoparticulate compositions of finasteride, dutasteride, or tamsulosin hydrochloride.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,582,285 for “Apparatus for sanitary wet milling;” U.S. Pat. No. 6,656,504 for “Nanoparticulate Compositions Comprising Amorphous Cyclosporine;” U.S. Pat. No. 6,742,734 for “System and Method for Milling Materials;” 6,745,962 for “Small Scale Mill and Method Thereof;” U.S. Pat. No. 6,811,767 for “Liquid droplet aerosols of nanoparticulate drugs;” and U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference. None of these patents describe nanoparticulate formulations of dutasteride or tamsulosin hydrochloride, although U.S. Patent Application No. 20020012675 A1 refers to controlled release formulations of finasteride. Moreover, none of the patents or patent publications describe injectable depot dosage forms of nanoparticulate dutasteride, tamsulosin hydrochloride, or finasteride.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Because finasteride, dutasteride, and tamsulosin hydrochloride are poorly water soluble, and because these drugs are useful in treating chronic conditions requiring long term and periodic treatment, improved dosage forms having increased bioavailability and prolonged activity are desirable. The present invention satisfies these needs.

### SUMMARY OF THE INVENTION

It is an object of the invention to provide compositions comprising nanoparticulate finesteride, nanoparticulate dutasteride, nanoparticulate tamsulosin hydrochloride, or a combination thereof, wherein the nanoparticulate finesteride, dutasteride, and/or tamsulosin hydrochloride have an effective average particle size of less than about 2000 nm. It is preferred that the active agent have adsorbed onto or associated with the surface of the active agent at least one surface stabilizer.

It is another object of the invention to provide formulations comprising a pharmaceutically effective nanoparticulate finesteride, dutasteride, and/or tamsulosin hydrochloride composition for the treatment of benign prostatic hyperplasia in mammals, in particular, in human patients.

It is a further object of the invention to provide methods of making a formulation for the treatment of benign prostatic hyperplasia.

It is a further object of the invention that the compositions of the invention be sufficiently stable so that a depot comprising one quantity or batch of the composition can provide continuous intramuscular or subcutaneous release of the composition to a patient or subject for up to about six months. In other embodiments of the invention, the release of the active agent is over alternative periods of time, such as up to about one week, up to about two weeks, up to about three weeks, up to about one month, up to about two months, up to about three months, up to about four months, or up to about five months.

In human therapy, it is important to provide a dosage form that delivers the required therapeutic amount of the active ingredient in vivo, and that renders the active ingredient bioavailable in a rapid and constant manner. The nanoparticulate formulations of the invention, which can be administered intramuscularly and subcutaneously, satisfy these needs.

The objectives are accomplished by a composition comprising at least one of finasteride, dutasteride, and tamsulosin hydrochloride which are collectively referred to in the application as the “active ingredient.” The formulation of the invention comprises the active ingredient having a surface stabilizer adsorbed on or associated with the surface of the active ingredient. In one embodiment of the invention, the surface stabilizer is a povidone polymer. In another embodiment of the invention, the active ingredient has an effective average particle size of less than about 2000 nm. In yet other embodiments of the invention, the effective average particle size of the nanoparticulate active ingredient is less than about 1000 nm, less than about 600 nm, less than about 450 nm, less than about 300 nm, less than about 250 nm, or less than about 100 nm.

The invention provides for compositions comprising concentrations of the active ingredient with rapid dissolution of the active ingredient upon administration.

In another aspect of the invention there is provided a method of preparing a nanoparticulate formulation of the active ingredient. The method comprises: (1) dispersing the active ingredient in a liquid dispersion medium; and (2) mechanically reducing the particle size of the active ingredient to an effective average particle size of less than about 2000 nm. A surface stabilizer, such as a povidone polymer with a molecular weight of less than about 40,000 daltons, can be added to the dispersion media either before, during, or after particle size reduction. Preferably, the pH of the liquid dispersion medium is maintained within the range of from about 3 to about 8 during the size reduction process.

Yet another aspect of the invention provides a method of treating a mammal, in particular, a human patient, for benign prostatic hyperplasia, comprising administering to the mammal a nanoparticulate active agent composition according to the invention. In yet another embodiment, the compositions of the invention are useful in treating alopecia.

It is to be understood that both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to the surprising and unexpected discovery that the pharmaceutical formulations or compositions of the invention for treatment of benign prostatic hyperplasia, or alopecia, can be intramuscularly or subcutaneously released continuously to a patient over a prolonged period of time, namely for up to about six months. The duration of release of the formulation is dependent upon the particle size of the active ingredient. The effective average particle size of the active ingredient is less than about 2000 nm, although smaller particle sizes are described herein, such less than about 600 nm, less than about 450 nm, less than about 300 nm, less than about 250 nm, or less than about 100 nm. The formulation comprises the nanoparticulate active ingredient with a surface stabilizer adsorbed onto or associated with the surface of the active ingredient particles. In one embodiment of the invention, the surface stabilizer is a povidone polymer having a molecular weight of not more than about 40,000 daltons.

The compositions comprise nanoparticles of at least one of finasteride, dutasteride and tamsulosin hydrochloride. Alternatively, the composition can be described as comprising nanoparticles of finasteride, dutasteride and tamsulosin hydrochloride, and mixtures thereof. The referenced nanoparticles are sometimes collectively referred to herein as the “active ingredient.”

Advantages of the nanoparticulate finasteride, dutasteride, tamsulosin hydrochloride, or combination thereof formulations of the invention over conventional forms of the drugs include, but are not limited to: (1) increased water solubility; (2) increased bioavailability; (3) smaller dosage form size or volume due to enhanced bioavailability; (4) lower therapeutic dosages due to enhanced bioavailability; (5) reduced risk of unwanted side effects; (6) enhanced patient convenience and compliance; (7) higher dosages possible without adverse side effects; (8) more effective BPH and/or alopecia treatment. A further advantage of the injectable nanoparticulate finasteride, dutasteride, tamsulosin hydrochloride, or combination thereof formulations of the invention over conventional forms of the drugs is the elimination of the need to use a solubilizing agent such as ethanol, polysorbates (e.g., polysorbate 80), alcohol, isopropyl alcohol, toluene, or derivatives thereof (e.g., butylated hydroxytoluene) to increase the solubility of the drug(s).

The present invention also includes nanoparticulate finasteride, dutasteride, tamsulosin hydrochloride, or combination thereof formulations together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like. A preferred dosage form is an injectable depot dosage form.

A. Definitions

The present invention is described herein using several definitions, as set forth below and throughout the application.

The term “effective average particle size of less than about 2000 nm,” as used herein means that at least 50% of the finasteride, dutasteride, or tamsulosin hydrochloride particles have a size, by weight, of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, a “stable” finasteride, dutasteride, or tamsulosin hydrochloride particle connotes, but is not limited to a finasteride, dutasteride, or tamsulosin hydrochloride particle with one or more of the following parameters: (1) the finasteride, dutasteride, or tamsulosin hydrochloride particles do not appreciably flocculate or agglomerate due to interparticle attractive forces or otherwise significantly increase in particle size over time; (2) the physical structure of the finasteride, dutasteride, or tamsulosin hydrochloride particles is not altered over time, such as by conversion from an amorphous phase to a crystalline phase; (3) the finasteride, dutasteride, or tamsulosin hydrochloride particles are chemically stable; and/or (4) where the finasteride, dutasteride, or tamsulosin hydrochloride has not been subject to a heating step at or above the melting point of the finasteride, dutasteride, or tamsulosin hydrochloride in the preparation of the nanoparticles of the invention.

The term “conventional” or “non-nanoparticulate” active agent or finasteride, dutasteride, or tamsulosin hydrochloride shall mean an active agent, such as finasteride, dutasteride, or tamsulosin hydrochloride, which is solubilized or which has an effective average particle size of greater than about 2000 nm. Nanoparticulate active agents as defined herein have an effective average particle size of less than about 2000 nm.

The phrase “poorly water soluble drugs” as used herein refers to drugs that have a solubility in water of less than about 30 mg/ml, less than about 20 mg/ml, less than about 10 mg/ml, or less than about 1 mg/ml.

As used herein, the phrase “therapeutically effective amount” means the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “modified release” as used herein in relation to the composition according to the invention or a coating or coating material or used in any other context means release which is not immediate release and is taken to encompass controlled release, sustained release, and delayed release.

The term “time delay” as used herein refers to the duration of time between administration of the composition and the release of finasteride, dutasteride, or tamsulosin hydrochloride from a particular component.

The term “lag time” as used herein refers to the time between delivery of active ingredient from one component and the subsequent delivery of the finasteride, dutasteride, or tamsulosin hydrochloride thereof from another component.

B. Features of the Nanoparticulate Finasteride, Dutasteride, or Tamsulosin hydrochloride Compositions

There are a number of enhanced pharmacological characteristics of the nanoparticulate finasteride, dutasteride, or tamsulosin hydrochloride compositions of the invention.

1. Increased Bioavailability

In one embodiment of the invention, the nanoparticulate finasteride, dutasteride, or tamsulosin hydrochloride formulations exhibit increased bioavailability at the same dose of the same active agent, and require smaller doses as compared to prior conventional finasteride, dutasteride, or tamsulosin hydrochloride formulations, such as PROSCAR®, AVODART®, or FLOMAX®.

A nanoparticulate finasteride, dutasteride, or tamsulosin hydrochloride dosage form requires less drug to obtain the same pharmacological effect observed with a conventional microcrystalline finasteride, dutasteride, or tamsulosin hydrochloride dosage form (e.g., PROSCAR®, AVODART®, or FLOMAX®). Therefore, the nanoparticulate finasteride, dutasteride, or tamsulosin hydrochloride dosage form has an increased bioavailability as compared to the conventional microcrystalline finasteride, dutasteride, or tamsulosin hydrochloride dosage form.

2. The Pharmacokinetic Profiles of the Finasteride, Dutasteride, or Tamsulosin Hydrochloride Compositions of the Invention are not Affected by the Fed or Fasted State of the Subject Ingesting the Compositions

In another embodiment of the invention described are nanoparticulate finasteride, dutasteride, or tamsulosin hydrochloride thereof compositions, wherein the pharmacokinetic profile of the finasteride, dutasteride, or tamsulosin hydrochloride is not substantially affected by the fed or fasted state of a subject ingesting the composition. This means that there is little or no appreciable difference in the quantity of drug absorbed or the rate of drug absorption when the nanoparticulate finasteride, dutasteride, or tamsulosin hydrochloride compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance with finasteride, dutasteride, or tamsulosin hydrochloride, an increase in the medical condition for which the drug is being prescribed may be observed—i.e., the prognosis for a BPH patient may worsen.

The invention also provides finasteride, dutasteride, or tamsulosin hydrochloride compositions having a desirable pharmacokinetic profile when administered to mammalian subjects. The desirable pharmacokinetic profile of the finasteride, dutasteride, or tamsulosin hydrochloride compositions preferably includes, but is not limited to: (1) a Cₘₐₓ for finasteride, dutasteride, or tamsulosin hydrochloride, when assayed in the plasma of a mammalian subject following administration, that is greater than the Cₘₐₓ for a non-nanoparticulate finasteride, dutasteride, or tamsulosin hydrochloride formulation (e.g., PROSCAR®, AVODART®, or FLOMAX®, administered at the same dosage; and/or (2) an AUC for finasteride, dutasteride, or tamsulosin hydrochloride, when assayed in the plasma of a mammalian subject following administration, that is greater than the AUC for a non-nanoparticulate finasteride, dutasteride, or tamsulosin hydrochloride formulation (e.g., PROSCAR®, AVODART®, or FLOMAX®), administered at the same dosage; and/or (3) a Tₘₐₓ for finasteride, dutasteride, or tamsulosin hydrochloride, when assayed in the plasma of a mammalian subject following administration, that is less than the Tₘₐₓ for a non-nanoparticulate finasteride, dutasteride, or tamsulosin hydrochloride formulation (e.g., PROSCAR®, AVODART®, or FLOMAX®), administered at the same dosage. The desirable pharmacokinetic profile, as used herein, is the pharmacokinetic profile measured after the initial dose of finasteride, dutasteride, or tamsulosin hydrochloride.

In one embodiment, a preferred finasteride, dutasteride, or tamsulosin hydrochloride composition exhibits in comparative pharmacokinetic testing with a non-nanoparticulate finasteride, dutasteride, or tamsulosin hydrochloride formulation (e.g., PROSCAR®, AVODART®, or FLOMAX®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate finasteride, dutasteride, or tamsulosin hydrochloride formulation (e.g., PROSCAR®, AVODART®, or FLOMAX®).

In another embodiment, the finasteride, dutasteride, or tamsulosin hydrochloride compositions of the invention exhibit in comparative pharmacokinetic testing with a non-nanoparticulate finasteride, dutasteride, or tamsulosin hydrochloride formulation (e.g., PROSCAR®, AVODART®, or FLOMAX®), administered at the same dosage, a Cmax which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cmax exhibited by the non-nanoparticulate finasteride, dutasteride, or tamsulosin hydrochloride formulation (e.g, PROSCAR®, AVODART®, or FLOMAX®).

In yet another embodiment, the finasteride, dutasteride, or tamsulosin hydrochloride compositions of the invention exhibit in comparative pharmacokinetic testing with a non-nanoparticulate finasteride, dutasteride, or tamsulosin hydrochloride formulation (e.g., PROSCAR®, AVODART®, or FLOMAX®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate finasteride, dutasteride, or tamsulosin hydrochloride formulation (e.g., PROSCAR®, AVODART®, or FLOMAX®).

3. Bioequivalency of the Finasteride, Dutasteride, or Tamsulosin hydrochloride Compositions of the Invention When Administered in the Fed Versus the Fasted State

The invention also encompasses a composition comprising a nanoparticulate finasteride, dutasteride, or tamsulosin hydrochloride in which administration of the composition to a subject in a fasted state is bioequivalent to administration of the composition to a subject in a fed state.

The difference in absorption of the compositions comprising the nanoparticulate finasteride, dutasteride, or tamsulosin hydrochloride when administered in the fed versus the fasted state, is preferably less than about 100%, less than about 95%, less than about 90%, less than about 85%, less than about 80%, less than about 75%, less than about 70%, less than about 65%, less than about 60%, less than about 55%, less than about 50%, less than about 45%, less than about 35%, less than about 35%, less than about 30%, less than about 25%, less than about 20%, less than about 15%, less than about 10%, less than about 5%, or less than about 3%.

In one embodiment of the invention, the invention encompasses a nanoparticulate finasteride, dutasteride, or tamsulosin hydrochloride wherein administration of the composition to a subject in a fasted state is bioequivalent to administration of the composition to a subject in a fed state, in particular as defined by Cₘₐₓ and AUC guidelines given by the U.S. Food and Drug Administration (USFDA) and the corresponding European regulatory agency (EMEA). Under USFDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

4. Dissolution Profiles of the Finasteride, Dutasteride, or Tamsulosin Hydrochloride Compositions of the Invention

In yet another embodiment of the invention, the finasteride, dutasteride, or tamsulosin hydrochloride compositions of the invention have unexpectedly dramatic dissolution profiles. Rapid dissolution of finasteride, dutasteride, or tamsulosin hydrochloride is preferable, as faster dissolution generally leads to faster onset of action and greater bioavailability. To improve the dissolution profile and bioavailability of finasteride, dutasteride, or tamsulosin hydrochloride, it is useful to increase the drug's dissolution so that it could attain a level close to 100%.

The finasteride, dutasteride, or tamsulosin hydrochloride compositions of the invention preferably have a dissolution profile in which within about 5 minutes at least about 20% of the finasteride, dutasteride, or tamsulosin hydrochloride composition is dissolved. In other embodiments of the invention, at least about 30% or at least about 40% of the finasteride, dutasteride, or tamsulosin hydrochloride composition is dissolved within about 5 minutes. In yet other embodiments of the invention, preferably at least about 40%, at least about 50%, at least about 60%, at least about 70%, or at least about 80% of the finasteride, dutasteride, or tamsulosin hydrochloride composition is dissolved within about 10 minutes. Finally, in another embodiment of the invention, preferably at least about 70%, at least about 80%, at least about 90%, or about at least about 100% of the finasteride, dutasteride, or tamsulosin hydrochloride composition is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

5. Redispersibility Profiles of the Finasteride, Dutasteride, or Tamsulosin hydrochloride Compositions of the Invention

In one embodiment of the invention, the finasteride, dutasteride, or tamsulosin hydrochloride compositions of the invention are formulated into solid dose forms which redisperse such that the effective average particle size of the redispersed finasteride, dutasteride, or tamsulosin hydrochloride particles is less than about 2 microns. This is significant, as if upon administration the nanoparticulate finasteride, dutasteride, or tamsulosin hydrochloride compositions did not redisperse to a nanoparticulate particle size, then the dosage form may lose the benefits afforded by formulating the finasteride, dutasteride, or tamsulosin hydrochloride into a nanoparticulate particle size.

Indeed, the nanoparticulate finasteride, dutasteride, or tamsulosin hydrochloride compositions of the invention benefit from the small particle size of the finasteride, dutasteride, or tamsulosin hydrochloride; if the finasteride, dutasteride, or tamsulosin hydrochloride does not redisperse into a small particle size upon administration, then “clumps” or agglomerated finasteride, dutasteride, or tamsulosin hydrochloride particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the nanoparticulate finasteride, dutasteride, or tamsulosin hydrochloride compositions of the invention exhibit dramatic redispersion of the nanoparticulate finasteride, dutasteride, or tamsulosin hydrochloride particles upon administration to a mammal, such as a human or animal, as demonstrated by reconstitution/redispersion in a biorelevant aqueous media such that the effective average particle size of the redispersed finasteride, dutasteride, or tamsulosin hydrochloride particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 N HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed finasteride, dutasteride, or tamsulosin hydrochloride particles of the invention (redispersed in an aqueous, biorelevant, or any other suitable media) have an effective average particle size of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

6. Finasteride, Dutasteride, or Tamsulosin Hydrochloride Compositions Used in Conjunction with Other Active Agents

The nanoparticulate finasteride, dutasteride, or tamsulosin hydrochloride compositions of the invention can additionally comprise one or more compounds useful in treating BPH or alopecia. The compositions of the invention can be co-formulated with such other active agents, or the compositions of the invention can be co-administered or sequentially administered in conjunction with such active agents.

C. Compositions

The invention provides compositions comprising nanoparticulate finasteride, dutasteride, or tamsulosin hydrochloride particles and at least one surface stabilizer. The surface stabilizers are preferably adsorbed onto or associated with the surface of the finasteride, dutasteride, or tamsulosin hydrochloride particles. Surface stabilizers useful herein do not chemically react with the finasteride, dutasteride, or tamsulosin hydrochloride particles or itself. Preferably, individual molecules of the surface stabilizer are essentially free of intermolecular cross-linkages. In another embodiment, the compositions of the invention can comprise two or more surface stabilizers.

The invention also includes nanoparticulate finasteride, dutasteride, or tamsulosin hydrochloride compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like. In certain embodiments of the invention, the nanoparticulate finasteride, dutasteride, or tamsulosin hydrochloride formulations are in an injectable form.

1. Active Ingredient

a. Finasteride

As used herein, the term “finesteride” includes analogs and salts thereof, and can be in a crystalline phase, an amorphous phase, a semi-crystalline phase, a semi-amorphous phase, or a mixture thereof. Finesteride may be present either in the form of one substantially optically pure enantiomer or as a mixture, racemic or otherwise, of enantiomers.

b. Dutasteride

As used herein, the term “dutasteride” includes analogs and salts thereof, and can be in a crystalline phase, an amorphous phase, a semi-crystalline phase, a semi-amorphous phase, or a mixture thereof. Dutasteride may be present either in the form of one substantially optically pure enantiomer or as a mixture, racemic or otherwise, of enantiomers.

C. Tamsulosin hydrochloride

As used herein, the term “tamsulosin hydrochloride” includes analogs and salts thereof, and can be in a crystalline phase, an amorphous phase, a semi-crystalline phase, a semi-amorphous phase, or a mixture thereof. Tamsulosin hydrochloride may be present either in the form of one substantially optically pure enantiomer or as a mixture, racemic or otherwise, of enantiomers.

2. Surface Stabilizers

Combinations of more than one surface stabilizer can be used in the finasteride, dutasteride, or tamsulosin hydrochloride formulations of the invention. In one embodiment of the invention, the finasteride, dutasteride, or tamsulosin hydrochloride formulation is an injectable formulation. Suitable surface stabilizers include, but are not limited to, known organic and inorganic pharmaceutical excipients. Such excipients include various polymers, low molecular weight oligomers, natural products, and surfactants. Surface stabilizers include nonionic, ionic, anionic, cationic, and zwitterionic surfactants. In one embodiment of the invention, a surface stabilizer for an injectable nanoparticulate finasteride, dutasteride, or tamsulosin hydrochloride formulation is a povidone polymer.

Representative examples of surface stabilizers include hydroxypropyl methylcellulose (now known as hypromellose), albumin, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Speciality Chemicals)); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂OH)₂ (Eastman Kodak Co.); decanoyl-N-methylglucamide; n-decyl (-D-glucopyranoside; n-decyl (-D-maltopyranoside; n-dodecyl (-D-glucopyranoside; n-dodecyl (-D-maltoside; heptanoyl-N-methylglucamide; n-heptyl-(-D-glucopyranoside; n-heptyl (-D-thioglucoside; n-hexyl (-D-glucopyranoside; nonanoyl-N-methylglucamide; n-noyl (-D-glucopyranoside; octanoyl-N-methylglucamide; n-octyl-(-D-glucopyranoside; octyl (-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate. Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulfate, lauryl dimethyl benzyl ammonium chloride or bromide, lauryl dimethyl (ethenoxy)4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336), POLYQUAT, tetrabutylammonium bromide, benzyl trimethylammonium bromide, choline esters (such as choline esters of fatty acids), benzalkonium chloride, stearalkonium chloride compounds (such as stearyltrimonium chloride and distearyldimonium chloride), cetyl pyridinium bromide or chloride, halide salts of quaternized polyoxyethylalkylamines, MIRAPOL and ALKAQUAT (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR1R2R3R4(+). For compounds of the formula NR1R2R3R4(+):

(i) none of R1-R4 are CH3;

(ii) one of R1-R4 is CH3;

(iii) three of R1-R4 are CH3;

(iv) all of R1-R4 are CH3;

(v) two of R1-R4 are CH3, one of R1-R4 is C6H5CH2, and one of R1-R4 is an alkyl chain of seven carbon atoms or less;

(vi) two of R1-R4 are CH3, one of R1-R4 is C6H5CH2, and one of R1-R4 is an alkyl chain of nineteen carbon atoms or more;

(vii) two of R1-R4 are CH3 and one of R1-R4 is the group C6H5(CH2)ₙ, where n>1;

(viii) two of R1-R4 are CH3, one of R1-R4 is C6H5CH2, and one of R1-R4 comprises at least one heteroatom;

(ix) two of R1-R4 are CH3, one of R1-R4 is C6H5CH2, and one of R1-R4 comprises at least one halogen;

(x)-two of R1-R4 are CH3, one of R1-R4 is C6H5CH2, and one of R1-R4 comprises at least one cyclic fragment;

(xi) two of R1-R4 are CH3 and one of R1-R4 is a phenyl ring; or

(xii) two of R1-R4 are CH3 and two of R1-R4 are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated herein by reference.

While applicants do not wish to be bound by theoretical mechanisms, it is believed that the surface stabilizer hinders the flocculation and/or agglomeration of the particles of the active ingredient by functioning as a mechanical or steric barrier between the particles, minimizing the close, interparticle approach necessary for agglomeration and flocculation.

Povidone Polymers

In one embodiment of the invention, the surface stabilizer is a povidone polymer. A povidone polymer surface stabilizer is particularly preferred when the compositions of the invention are formulated into injectable dosage forms. Povidone polymers, also known as polyvidon(e), povidonum, PVP, and polyvinylpyrrolidone, are sold under the trademarks Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. To be useful as a surface stabilizer for the active ingredient to be administered to a mammal via injection, the povidone polymer must have a molecular weight of not greater than about 40,000 daltons, as a molecular weight of greater than 40,000 daltons would have difficulty clearing the body.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° C. to form γ-butyrolactone; and (3) reacting γ-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10^{th} Edition, pp. 7581 (Merck & Co., Rahway, N.J., 1983).

It is preferred that the nanoparticulate active agent/povidone polymer pharmaceutical formulation of the invention has a pH of between about 6 to about 7.

The manufacturing process for povidone polymers produces polymers comprising molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering. Table 1 provides molecular weight data for several commercially available povidone polymers, all of which are soluble.

TABLE 1

| Povidone | K-Value | Mv (Daltons)** | Mw (Daltons)** | Mn (Daltons)** |
|---|---|---|---|---|
| Plasdone ® C-15 | 17 ± 1 | 7,000 | 10,500 | 3,000 |
| Plasdone ® C-30 | 30.5 ± 1.5 | 38,000 | 62,500* | 16,500 |
| Kollidon ® 12 PF | 11-14 | 3,900 | 2,000-3,000 | 1,300 |
| Kollidon ® 17 PF | 16-18 | 9,300 | 7,000-11,000 | 2,500 |
| Kollidon ® 25 | 24-32 | 25,700 | 28,000-34,000 | 6,000 |

*Because the molecular weight is greater than 40,000 daltons, this povidone polymer is not useful as a surface stabilizer for a drug compound to be administered parenterally (i.e., injected).
**Mv is the viscosity-average molecular weight, Mn is the number-average molecular weight, and Mw is the weight average molecular weight. Mw and Mn were determined by light scattering and ultra-centrifugation, and Mv was determined by viscosity measurements.

Based on the data provided in Table 1, exemplary preferred commercially available povidone polymers include, but are not limited to, Plasdone® C-15, Kollidon® 12 PF, Kollidon® 17 PF, and Kollidon® 25.

3. Finasteride, Dutasteride and Tamsulosin Hydrochloride Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

By “an effective average particle size of less than about 2000 nm” it is meant that at least 50% of the finasteride, dutasteride or tamsulosin hydrochloride particles, by weight, have a particle size of less than about 2000 nm when measured by the above-noted techniques. In other embodiments of the invention, the finasteride, dutasteride or tamsulosin hydrochloride particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 mm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 mm, less than about 150 nm, less than about 100 nm, less than about 75 nm, or less than about 50 nm, as measured by light-scattering methods, microscopy, or other appropriate methods.

In another embodiment of the invention, the compositions of the invention are in an injectable dosage form and the finasteride, dutasteride or tamsulosin hydrochloride particles preferably have an effective average particle size of less than about 1000 nm, less than about 900 nm, less than about 800 nm, less than about 700 nm, less than about 650 mm, less than about 600 nm, less than about 550 nm, less than about 500 nm, less than about 450 nm, less than about 400 nm, less than about 350 nm, less than about 300 mm, less than about 250 nm, less than about 200 nm, less than about 150 nm, less than about 100 nm, less than about 75 nm, or less than about 50 nm, as measured by light-scattering methods, microscopy, or other appropriate methods. Injectable compositions can comprise finasteride, dutasteride or tamsulosin hydrochloride particles having an effective average particle size of greater than about 1 micron, up to about 2 microns.

With reference to the effective average particle size, in other embodiments of the invention, at least about 60%, at least about 70%, at least about 80%, at least about 90%, at least about 95%, or at least about 99% of the finasteride, dutasteride or tamsulosin hydrochloride particles have a particle size less than the effective average particle size. In particularly preferred embodiments essentially all of the particles have a size less of than about 2000 nm.

In the invention, the value for D50 of a nanoparticulate finasteride, dutasteride or tamsulosin hydrochloride composition is the particle size below which 50% of the finasteride, dutasteride or tamsulosin hydrochloride particles fall, by weight. Similarly, D90 is the particle size below which 90% of the finasteride, dutasteride or tamsulosin hydrochloride particles fall, by weight.

4. Concentration of Nanoparticulate Finasteride, Dutasteride and Tamsulosin Hydrochloride and Surface Stabilizers

The relative amounts of finasteride, dutasteride or tamsulosin hydrochloride and one or more surface stabilizers can vary widely. The optimal amount of the individual components depends, for example, upon physical and chemical attributes of the surface stabilizer(s) and the active agent selected, such as the hydrophilic lipophilic balance (HLB), melting point, and the surface tension of water solutions of the stabilizer, etc.

Preferably, the concentration of finasteride, dutasteride or tamsulosin hydrochloride can vary from about 99.5% to about 0.001%, from about 95% to about 0.1%, or from about 90% to about 0.5%, by weight, based on the total combined weight of finasteride, dutasteride or tamsulosin hydrochloride and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of finasteride, dutasteride, or tamsulosin hydrochloride and at least one surface stabilizer, not including other excipients.

5. Other Pharmaceutical Excipients

Pharmaceutical compositions of the invention may also comprise one or more binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are well known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, and quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples, such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

6. Injectable Nanoparticulate Finasteride, Dutasteride, or Tamsulosin hydrochloride Formulations

In one embodiment of the invention, provided are injectable nanoparticulate finasteride, dutasteride or tamsulosin hydrochloride formulations that can comprise high concentrations in low injection volumes, with rapid dissolution upon administration. Exemplary compositions comprise, based on % w/w:

| | |
|---|---|
| finasteride, dutasteride or | 5-50% |
| tamsulosin hydrochloride | |
| Surface stabilizer | 0.1-50% |
| preservatives | 0.05-0.25% |
| pH adjusting agent | pH about 6 to about 7 |
| water for injection | q.s. |

Exemplary preservatives include methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

In one embodiment, the invention is directed to the unexpected discovery that nanoparticulate finasteride, dutasteride, tamsulosin hydrochloride, or a combination thereof can be successfully utilized in injectable depot dosage forms. The injectable depot formulation provides release of the active agent over a prolonged period of time, up to about 6 months. In another embodiment of the invention, the release from the injectable depot dosage form can be up to about 1 week, up to about 2 weeks, up to about 3 weeks, up to about 4 weeks, up to about 5 weeks, up to about 1 month, up to about 2 months, up to about 3 months, up to about 4 months, up to about 5 months, or up to about 6 months.

Current formulations of finasteride, dutasteride, tamsulosin hydrochloride, such as PROSCAR®, AVODART®, and FLOMAX®, are oral dosage forms that require frequent periodic, such as daily, administration. Many patients do not conform to the suggested periodic or daily dosage regimen. Moreover, some studies suggest that up to a third of all patients fail to follow the prescribed dosing schedule for prescribed medicines. Thus, dosage forms of finasteride, dutasteride, tamsulosin hydrochloride which eliminate the need for patient compliance regarding periodic, or daily, administration are highly desirable. Conventional forms of finasteride, dutasteride, tamsulosin hydrochloride, such as PROSCAR®, AVODART®, and FLOMAX®, cannot be formulated into injectable dosage forms. Prior to the present invention it was not know that finasteride, dutasteride, tamsulosin hydrochloride could be successfully formulated into an injectable depot dosage form by formulating the active ingredients into a nanoparticulate particle size.

U.S. Pat. No. 6,238,693 B1 to Luther et al., which is incorporated herein by reference, illustrates in FIGS. 5 and 6 the use of a drug depot for the release of a drug to a patient over time.

D. Method of Making Formulations Comprising the Active Ingredient

In another aspect of the invention there is provided a method of preparing the nanoparticulate finasteride, dutasteride or tamsulosin hydrochloride formulations of the invention. Nanoparticulate finasteride, dutasteride or tamsulosin hydrochloride formulations can be made using any suitable method known in the art such as, for example, milling, homogenization, precipitation, or supercritical fluid particle generation techniques.

An exemplary method comprises: (1) dispersing the active ingredient in a liquid dispersion medium in which the active ingredient is poorly soluble; and (2) mechanically reducing the particle size of the active ingredient to an effective average particle size of less than about 2000 nm. A surface stabilizer, such as a povidone polymer with a molecular weight of less than about 40,000 daltons, can be added to the dispersion media either before, during, or after particle size reduction of the active ingredient. The pH of the liquid dispersion medium is preferably maintained within the range of from about 5.0 to about 7.5 during the size reduction process. Preferably, the dispersion medium used for the size reduction process is aqueous, although any dispersion media in which the active ingredient is poorly soluble can be used, such as safflower oil, ethanol, t-butanol, glycerin, polyethylene glycol (PEG), hexane, or glycol.

Effective methods of providing mechanical force for particle size reduction of the active ingredient include ball milling, media milling, and homogenization, for example, with a Microfluidizer® machine (Microfluidics Corp.). Ball milling is a low energy milling process that uses milling media, drug, stabilizer, and liquid. The materials are placed in a milling vessel that is rotated at optimal speed such that the media cascades and reduces the particle size by impaction. The media used must have a high density as the energy for the particle reduction is provided by gravity and the mass of the attrition media.

Media milling is a high energy milling process. The active ingredient, stabilizer, and liquid are placed in a reservoir and recirculated in a chamber containing media and a rotating shaft/impeller. The rotating shaft agitates the media, which subjects the active ingredient and stabilizer to impaction and sheer forces, thereby reducing their size.

Homogenization is a technique that does not use milling media. The active ingredient, stabilizer, and liquid (or active ingredient and liquid with the stabilizer added after particle size reduction) are stream propelled into a process zone, which in the Microfluidizer® machine is called the Interaction Chamber. The product to be treated is inducted into the pump, and then forced out. The priming valve of the Microfluidizer® machine purges air out of the pump. Once the pump is filled with product, the priming valve is closed and the product is forced through the interaction chamber. The geometry of the interaction chamber produces powerful forces of sheer, impact, and cavitation, which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® machine also provides a heat exchanger to allow cooling of the product. U.S. Pat. No. 5,510,118 to Bosch et al., which is specifically incorporated herein by reference, refers to a process using a Microfluidizer® resulting in sub 400 nm particles.

Using a particle size reduction method, the particle size of the active ingredient is reduced to an effective average particle size of less than about 2000 mm. The particles of the active ingredient can be reduced in size in the presence of a surface stabilizer, such as a povidone polymer, or the surface stabilizer can be added to the dispersion of the active ingredient during or after particle size reduction.

The active ingredient can be added to a liquid medium in which it is essentially insoluble to form a premix. The concentration of the active ingredient in the liquid medium can vary from about 5 to about 60%, and preferably is from about 15 to about 50% (w/v), and more preferably, about 20 to about 40%. The surface stabilizer can be present in the premix or it can be added to the dispersion of the active ingredient following particle size reduction. The concentration of the surface stabilizer can vary from about 0.1 to about 50%, and preferably is from about 0.5 to about 20%, and more preferably, from about 1 to about 10%, by weight.

The premix can be used directly by subjecting it to mechanical means to reduce the average particle size of the active ingredient in the dispersion to less than about 2000 nm. It is preferred that the premix be used directly when a ball mill is used for attrition. Alternatively, the active ingredient and the surface stabilizer can be dispersed in the liquid medium using suitable agitation, e.g., a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the particle size of the active ingredient conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1,000 centipoise, and for ball milling the apparent viscosity of the premix is preferably from about 1 up to about 100 centipoise. Such ranges tend to afford an optimal balance between efficient particle size reduction and media erosion.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The particles of the active ingredient can be reduced in size at a temperature which does not significantly degrade it. Processing temperatures of less than about 30° C. to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

1. Grinding Media

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of material for the grinding media is not believed to be critical. Zirconium oxide, such as 95% ZrO stabilized with magnesia, zirconium silicate, ceramic, stainless steel, titania, alumina, 95% ZrO stabilized with yttrium, and glass grinding media are exemplary grinding materials.

The grinding media can comprise particles that are preferably substantially spherical in shape, e.g., beads, consisting essentially of polymeric resin or glass or Zirconium Silicate or other suitable compositions. Alternatively, the grinding media can comprise a core having a coating of a polymeric resin adhered thereon.

The grinding media can comprise particles that are preferably substantially spherical in shape, e.g., beads, consisting essentially of polymeric resin. Alternatively, the grinding media can comprise a core having a coating of a polymeric resin adhered thereon. The polymeric resin can have a density from about 0.8 to about 3.0 g/cm³.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably, from about 0.03 to about 1 mm in size.

In a preferred grinding process the particles are made continuously. Such a method comprises continuously introducing the active ingredient into a milling chamber, contacting the active ingredient with grinding media while in the chamber to reduce the particle size, and continuously removing the nanoparticulate active ingredient from the milling chamber.

The grinding media is separated from the milled nanoparticulate active ingredient using conventional separation techniques, in a secondary process such as by simple filtration, sieving through a mesh filter or screen, and the like. Other separation techniques such as centrifugation may also be employed.

2. Sterile Product Manufacturing

Development of the composition to be administered intramuscularly or subcutaneously requires the production of a sterile product. The manufacturing process of the present invention is similar to typical known manufacturing processes for sterile suspensions. A typical sterile suspension manufacturing process flowchart is as follows:

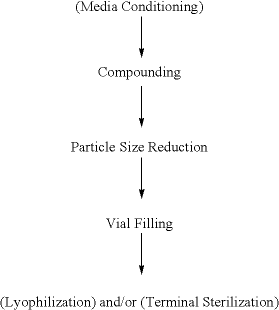

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

E. Method of Treatment

Yet another aspect of the present invention provides a method of treating a mammal, in particular, a human patient, requiring treatment for benign prostatic hyperplasia or alopecia comprising to the mammal the nanoparticulate finasteride, dutasteride, tamsulosin hydrochloride, or a combination thereof formulation of the invention. A preferred administration method is intramuscular or subcutaneous administration. Particularly advantageous features of the invention include that the pharmaceutical formulation of the invention exhibits unexpectedly prolonged release, dependent upon particle size, from the administration site. In addition, the formulation of the invention can provide a high concentration in a small volume to be intramuscularly or subcutaneously administered.

The compositions of the invention can be formulated: (a) for administration selected from the group consisting of oral, pulmonary, rectal, opthalmic, colonic, parenteral, intracisternal, intravaginal, intraperitoneal, local, buccal, nasal, and topical administration; (b) into a dosage form selected from the group consisting of liquid dispersions, solid dispersions, liquid-filled capsule, gels, aerosols, ointments, creams, lyophilized formulations, tablets, capsules, multi-particulate filled capsule, tablet composed of multi-particulates, compressed tablet, and a capsule filled with enteric-coated beads of a docetaxel or analogue thereof, (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The pharmaceutical composition of the invention is effective for at least six months with proper handling. In a preferred embodiment of the invention, a portion of the pharmaceutical formulation representing a patient dosage for a period of time is maintained in a depot, i.e., a fixed or transportable repository of sufficient size to allow constant release of the composition to a patient for up to six months. Such long-term release of the active ingredient would improve patient compliance and, therefore, the efficacy of the treatment.

In human therapy, it is important to provide a finasteride, dutasteride, tamsulosin hydrochloride, or a combination thereof dosage form that delivers the required therapeutic amount of the drug in vivo, and that renders the drug bioavailable in a constant manner. Thus, another aspect of the present invention provides a method of treating a mammal, including a human, requiring alopecia or BPH treatment comprising administering to the mammal the nanoparticulate finasteride, dutasteride, tamsulosin hydrochloride, or a combination thereof formulation of the invention.

In yet another embodiment of the invention, the nanoparticulate finasteride, dutasteride, tamsulosin hydrochloride, or a combination thereof composition of the invention can be administered at significantly higher doses as compared to the comparable non-nanoparticulate finasteride, dutasteride, or tamsulosin hydrochloride formulation.

In one embodiment of the invention, the nanoparticulate finasteride, dutasteride, tamsulosin hydrochloride, or a combination thereof composition, including an injectable composition, is free of a solubilizing agent, such as ethanol, polysorbates (e.g., polysorbate 80), alcohol, isopropyl alcohol, toluene, or derivatives thereof (e.g., butylated hydroxytoluene) to increase the solubility of the drug(s). In addition, when formulated into an injectable formulation, the compositions of the invention can provide a high concentration in a small volume to be injected. Injectable finasteride, dutasteride, tamsulosin hydrochloride, or a combination thereof compositions of the invention can be administered in an injectable depot, bolus injection, or with a slow infusion over a suitable period of time.

One of ordinary skill will appreciate that effective amounts of a finasteride, dutasteride, tamsulosin hydrochloride, or a combination thereof can be determined empirically and can be employed in pure form or, where such forms exist, in pharmaceutically acceptable salt, ester, or prodrug form. Actual dosage levels of finasteride, dutasteride, tamsulosin hydrochloride, or a combination thereof in the injectable or other dosage forms of the invention may be varied to obtain an amount of finasteride, dutasteride, tamsulosin hydrochloride, or a combination thereof that is effective to obtain a desired therapeutic response for a particular composition and method of administration. The selected dosage level therefore depends upon the desired therapeutic effect, the route of administration, the potency of the administered finasteride, dutasteride, or tamsulosin hydrochloride, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXAMPLE 1

The purpose of this example was to prepare a nanoparticulate formulation of finasteride.

An aqueous dispersion of 5% (w/w) finasteride (Form III, Supplier: Camida, Tower House, New Quay, Clonmel, County Tipperary, Ireland; Manufacturer: Dr. Reddy's, Unit-II, Factory Plot No. 110 & 111, S.V. Co-op., Industrial Estate, Bollaram, Narsapur Tq., Medak Dist., A.P.), combined with 1.5% (w/w) Tween 80 (Polyoxyethylene Sorbitan Fatty acid Esters), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min, and then harvested using 21 gauge syringe.

Following milling, the sample was paste-like in texture. Thus, microscopy observation and particle size analysis of the milled finasteride particles could not be performed. This example demonstrates that Tween 80, at the concentration of surface stabilizer and drug used, does not produce a stable nanoparticulate composition of finasteride.

### EXAMPLE 2

The purpose of this example was to prepare a nanoparticulate formulation of finasteride.

An aqueous dispersion of 5% (w/w) finasteride, combined with 1.25% (w/w) Plasdone C-15 (Povidone K15.5-17.5) and 0.05% (w/w) deoxycholate acid sodium salt, was milled in a 10 ml chamber of a NanoMill®0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 3500 rpms for 60 min. Subsequently, the same mixture was further milled at a speed of 4000 rpms for 30 min before sample 2 was harvested. The samples were harvested using 21 gauge syringe after milling, demonstrating that the samples can be used in injectable formulations.

Microscopy of the milled sample 2, using a Lecia DM5000B and Lecia CTR 5000 light source (Laboratory Instruments and Supplies Ltd., Ashbourne Co., Meath, Ireland), showed well dispersed discrete particles. There were also some larger “block-like” shaped particles present. Brownian motion was clearly evident for all particles with no signs of flocculation.

Following milling and optional 60 seconds of sonication (noted in Table 1 below), the particle size of the finasteride particles in both samples was measured, in deionized distilled water, using a Horiba LA 910 particle size analyzer. The particle size measured is shown in Table 2, below.

TABLE 2

| | Mean | D50 | | |
|---|---|---|---|---|
| | Particle | Particle | D90 Particle | D95 Particle |
| Samples | Size (nm) | Size (nm) | Size (nm) | Size (nm) |
| Sample 1 without | 510 | 459 | 787 | 957 |
| sonication | | | | |
| Sample 1 with | 503 | 457 | 767 | 926 |
| sonication | | | | |
| Sample 2 without | 447 | 416 | 663 | 773 |
| sonication | | | | |
| Sample 2 with | 444 | 414 | 658 | 762 |
| sonication | | | | |

The results demonstrate the successful preparation of stable, nanoparticulate compositions of finesteride, as the D50 particle sizes of the composition produced were less than about 2000 nm. Moreover, the particle size of the two samples did not vary significantly, demonstrating that the first round of milling was sufficient to generate a successful preparation.

### EXAMPLE 3

The purpose of this example was to prepare a nanoparticulate formulation of finasteride.

An aqueous dispersion of 5% (w/w) finasteride, combined with 1.25% (w/w) HPC-SL (hydroxypropyl cellulose) and 0.05% (w/w) docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 4000 rpms for 60 min. Subsequently, the same mixture was further milled at a speed of 2500 rpms for 45 min before sample 2 was harvested. The samples were harvested using 21 gauge syringe after milling, demonstrating that the samples can be used in injectable formulations.

Microscopy of both of the milled samples, using a Lecia DM5000B and Lecia CTR 5000 light source (Laboratory Instruments and Supplies Ltd., Ashbourne Co., Meath, Ireland), showed well dispersed discrete particles with clear evidence of Brownian motion. Sample 1 contained some aggregated crystals and “unmilled” material. There were isolated crystals of “unmilled” material in sample 2 as well. Isolated pockets of aggregated material were also visible in sample 2, which may suggest that slight flocculation had occurred.

Following milling and optional 60 second sonication, the particle size of the finasteride particles in both samples was measured, in deionized distilled water, using a Horiba LA 910 particle size analyzer. The particle size measured is shown in Table 3, below.

TABLE 3

| | | D50 | D90 | |
|---|---|---|---|---|
| | Mean Particle | Particle | Particle | D95 Particle |
| Samples | Size (nm) | Size (nm) | Size (nm) | Size (nm) |
| Sample 1 without | 1950 | 330 | 6270 | 12422 |
| sonication | | | | |
| Sample 1 with | 519 | 305 | 988 | 1976 |
| sonication | | | | |
| Sample 2 without | 437 | 331 | 682 | 1088 |
| sonication | | | | |
| Sample 2 with | 386 | 329 | 598 | 797 |
| sonication | | | | |

The results demonstrate the successful preparation of stable, nanoparticulate compositions of finesteride, as the D50 particle sizes of the composition produced were less than about 2000 nm. However, sample 1 appears less favorable than sample 2, as large aggregates may be present. Such large aggregates are not desirable in injectable formulations. Moreover, such large aggregates can result in inconsistent bioavailability when formulated in other types of dosage forms. Thus, the longer milling period may be necessary to generate a successful preparation for this particular combination of surface stabilizer and finesteride, at the particular drug and surface stabilizer concentrations utilized.

### EXAMPLE 4

The purpose of this example was to prepare a nanoparticulate formulation of finasteride.

An aqueous dispersion of 5% (w/w) finasteride, combined with 1.25% (w/w) Plasdone K-17 (Povidone K17) and 0.05% (w/w) benzalkonium chloride, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 2500 rpms for 60 min. Sample 2 was harvested after the same mixture was milled for an additional 60 min at the same speed. Subsequently, the same mixture was further milled at a speed of 3500 rpm for 30 min before sample 3 was harvested. The samples were harvested using 21 gauge syringe after milling, demonstrating that the samples can be used in injectable formulations.

Microscopy of sample 1, using a Lecia DM5000B and Lecia CTR 5000 light source (Laboratory Instruments and Supplies Ltd., Ashbourne Co., Meath, Ireland), showed well dispersed discrete particles with clear evidence of Brownian motion, although a lot of “rod-like” crystals were also present. These crystals could represent crystal growth or “un-milled” material.

Following milling and optional 60 seconds sonication, the particle size of the finasteride particles in all three samples was measured, in deionized distilled water, using a Horiba LA 910 particle size analyzer. The particle size measured is shown in Table 4, below.

TABLE 4

| | | | D90 | D95 |
|---|---|---|---|---|
| | Mean Particle | D50 Particle | Particle | Particle |
| Samples | Size (nm) | Size (nm) | Size (nm) | Size (nm) |
| Sample 1 without | 1926 | 1186 | 4722 | 6208 |
| sonication | | | | |
| Sample 1 with | 1843 | 1121 | 4497 | 5970 |
| sonication | | | | |
| Sample 2 without | 1231 | 565 | 3197 | 4632 |
| sonication | | | | |
| Sample 2 with | 1203 | 558 | 3103 | 4522 |
| sonication | | | | |
| Sample 3 without | 1252 | 706 | 2933 | 3961 |
| sonication | | | | |
| Sample 3 with | 1218 | 689 | 2846 | 3850 |
| sonication | | | | |

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples. Moreover, the particle size of sample 2 and sample 3 did not vary significantly, demonstrating that the milling time periods used for these samples were sufficient to generate a successful preparation.

### EXAMPLE 5

The purpose of this example was to prepare a nanoparticulate formulation of finasteride.

An aqueous dispersion of 5% (w/w) finasteride, combined with 1.5% (w/w) Pluronic F108 (Poloxamer 308), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 3500 rpms for 30 min. Subsequently, the same mixture was further milled at the same speed for an additional 60 min before sample 2 was harvested. The samples were harvested using 21 gauge syringe after milling, demonstrating that the samples can be used in injectable formulations.

Following milling and optional 60 seconds sonication, the particle size of the finasteride particles in both samples was measured, in deionized distilled water, using a Horiba LA 910 particle size analyzer. The particle size of sample 2 was re-measured under the same parameters three days after the sample preparation. The particle size measured is shown in Table 5, below.

TABLE 5

| | | D50 | D90 | |
|---|---|---|---|---|
| | Mean Particle | Particle | Particle | D95 Particle |
| Samples | Size (nm) | Size (nm) | Size (nm) | Size (nm) |
| Sample 1 without | 1684 | 1471 | 2955 | 3702 |
| sonication | | | | |
| Sample 1 with | 1655 | 1463 | 2887 | 3569 |
| sonication | | | | |
| Sample 2 without | 1404 | 1182 | 2546 | 3265 |
| sonication | | | | |
| Sample 2 with | 1343 | 1156 | 2422 | 3029 |
| sonication | | | | |
| Sample 2* | 1882* | 1537* | 3446* | 4464* |
| without | | | | |
| sonication | | | | |
| Sample 2* with | 1727* | 1489* | 3007* | 3773* |
| sonication | | | | |

*The re-measurement data is indicated by “*” in this table.

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples. Additionally, the particle size of the two samples did not vary significantly, demonstrating that the milling time period used was sufficient to generate a successful preparation. The particle size of sample 2 was increased in re-measurement performed three days after sample preparation, demonstrating possible crystal growth in the sample.

### EXAMPLE 6

The purpose of this example was to prepare a nanoparticulate formulation of finasteride.

An aqueous dispersion of 5% (w/w) finasteride, combined with 1.25% (w/w) Lutrol F68 (Poloxamer 188) and 0.05% w/w docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load).). Sample 1 was harvested after the mixture was initially milled at a speed of 3500 rpms for 90 min. Subsequently, the same mixture was further milled at a speed of 2500 rpms for 30 min before sample 2 was harvested. The samples were harvested using 21 gauge syringe after milling, demonstrating that the samples can be used in injectable formulations.

Microscopy of sample 2, using a Lecia DM5000B and Lecia CTR 5000 light source (Laboratory Instruments and Supplies Ltd., Ashbourne Co., Meath, Ireland), showed presence of nano-particles and evidence of Brownian motion, although severe flocculation was observed with more than 50% of the slide showing aggregation. There was no sign of crystal growth or “un-milled” material.

Following milling and optional 60 seconds sonication, the particle size of the finasteride particles in both samples was measured, in deionized distilled water, using a Horiba LA 910 particle size analyzer. The particle size measured is shown in Table 6, below.

TABLE 6

| | | D50 | D90 | |
|---|---|---|---|---|
| | Mean Particle | Particle | Particle | D95 Particle |
| Samples | Size (nm) | Size (nm) | Size (nm) | Size (nm) |
| Sample 1 without | 4160 | 2932 | 9973 | 12598 |
| sonication | | | | |
| Sample 1 with | 540 | 458 | 915 | 1144 |
| sonication | | | | |
| Sample 2 without | 1976 | 1152 | 4914 | 6240 |
| sonication | | | | |
| Sample 2 with | 397 | 371 | 584 | 675 |
| sonication | | | | |

The results demonstrate that both of the samples likely contained aggregates of finesteride particles, as the samples with sonication had significantly different particle sizes as compared to the samples without sonication. Such large aggregates are not desirable in injectable formulations or other types of dosage forms due to inconsistent bioavailability. The particle size of sample 2 was more favorable than that of sample 1, demonstrating that the second round of milling was necessary to generate a successful preparation.

### EXAMPLE 7

The purpose of this example was to prepare a nanoparticulate formulation of finasteride.

An aqueous dispersion of 5% (w/w) finasteride, combined with 1.25% (w/w) Pharmacoat 603, and 0.05% w/w docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 3500 rpms for 90 min. Subsequently, the same mixture was further milled at a speed of 4500 rpms for 60 min before sample 2 was harvested. The samples were harvested using 21 gauge syringe after milling, demonstrating that the samples can be used in injectable formulations.

Microscopy of sample 2, using a Lecia DM5000B and Lecia CTR 5000 light source (Laboratory Instruments and Supplies Ltd., Ashbourne Co., Meath, Ireland), showed well dispersed nano-particles with evident Brownian motion, although the majority of nano-particles were “rod-like” in shape. There was no sign of flocculation.

Following milling and optional 60 seconds sonication, the particle size of the finasteride particles in both samples was measured, in deionized distilled water, using a Horiba LA 910 particle size analyzer. The particle size measured is shown in Table 7 below.

TABLE 7

| | | D50 | D90 | |
|---|---|---|---|---|
| | Mean Particle | Particle | Particle | D95 Particle |
| Samples | Size (nm) | Size (nm) | Size (nm) | Size (nm) |
| Sample 1 without | 1175 | 947 | 2263 | 2840 |
| sonication | | | | |
| Sample 1 with | 1115 | 921 | 2114 | 2594 |
| sonication | | | | |
| Sample 2 without | 616 | 478 | 1143 | 1496 |
| sonication | | | | |
| Sample 2 with | 585 | 470 | 1059 | 1362 |
| sonication | | | | |

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples. Moreover, the D50 particle size of both samples was less than about 2000 nm, demonstrating that the time period used in the first round of milling was sufficient to generate a successful preparation.

### EXAMPLE 8

The purpose of this example was to prepare a nanoparticulate formulation of finasteride.

An aqueous dispersion of 5% (w/w) finasteride, combined with 1.25% (w/w) Plasdone S-630 (Copovidone K25-34) and 0.05% (w/w) lauryl sulfate (sodium lauryl sulfate), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 3500 rpms for 90 min. The sample was harvested using 21 gauge syringe after milling, demonstrating that the sample can be used in injectable formulations.

Microscopy of the milled sample, using a Lecia DM5000B and Lecia CTR 5000 light source (Laboratory Instruments and Supplies Ltd., Ashbourne Co., Meath, Ireland), showed well dispersed discrete particles with clear evidence of Brownian motion. There were also isolated particles of “unmilled” material visible, exhibiting signs of crystal growth. There was no evidence of aggregation present.

Following milling and optional 60 seconds of sonication, the particle size of the finasteride particles was measured, in deionized distilled water, using a Horiba LA 910 particle size analyzer. The particle size measured is shown in Table 8 below.

TABLE 8

| | Mean Particle | D50 Particle | D90 Particle | D95 Particle |
|---|---|---|---|---|
| Samples | Size (nm) | Size (nm) | Size (nm) | Size (nm) |
| Sample | 353 | 318 | 506 | 622 |
| without | | | | |
| sonication | | | | |
| Sample with | 354 | 317 | 508 | 634 |
| sonication | | | | |

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples.

### EXAMPLE 9

The purpose of this example was to prepare a nanoparticulate formulation of finasteride.

An aqueous dispersion of 5% (w/w) finasteride, combined with 2% (w/w) HPC-SL (hydrocypropyl cellulose, super low viscosity), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 3500 rpms for 60 min. Subsequently, the same mixture was milled at the same speed for an additional 60 min before sample 2 was harvested. The samples were harvested using 21 gauge syringe after milling, demonstrating that the samples can be used in injectable formulations.

Microscopy of the milled sample 2, using a Lecia DM5000B and Lecia CTR 5000 light source (Laboratory Instruments and Supplies Ltd., Ashbourne Co., Meath, Ireland), showed well dispersed nano-particles with clear evidence of Brownian motion. There was no sign of crystal growth and flocculation.

Following milling and optional 60 seconds of sonication, the particle size of the finasteride particles in both samples was measured, in deionized distilled water, using a Horiba LA 910 particle size analyzer. The particle size measured is shown in Table 9 below.

TABLE 9

| | | D50 | D90 | |
|---|---|---|---|---|
| | Mean Particle | Particle | Particle | D95 Particle |
| Samples | Size (nm) | Size (nm) | Size (nm) | Size (nm) |
| Sample 1 without | 10513 | 6918 | 25032 | 31010 |
| sonication | | | | |
| Sample 1 with | 6085 | 631 | 13407 | 26241 |
| sonication | | | | |
| Sample 2 without | 292 | 285 | 389 | 431 |
| sonication | | | | |
| Sample 2 with | 292 | 286 | 387 | 428 |
| sonication | | | | |

The results demonstrate the successful preparation of stable, nanoparticulate compositions of finesteride from sample 2 or from sample 1 subjected to 60-second sonication, as the D50 particle size of the compositions produced was less than about 2000 nm. Moreover, the particle size measurements in sample 2 did not change significantly following sonication, demonstrating that aggregates of finesteride were not present in the sample after the longer milling periods.

### EXAMPLE 10

The purpose of this example was to prepare a nanoparticulate formulation of finasteride.

An aqueous dispersion of 5% (w/w) finasteride, combined with 1% (w/w) Lutrol F108 (Poloxamer 338) and 1% (w/w) Tween 80 (polyoxyethylene sorbitan fatty acid esters), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 3500 rpms for 60 min. The sample was harvested using 21 gauge syringe after milling, demonstrating that the sample can be used in injectable formulations.

Microscopy of the milled sample, using a Lecia DM5000B and Lecia CTR 5000 light source (Laboratory Instruments and Supplies Ltd., Ashbourne Co., Meath, Ireland), showed flocculation and “unmilled” crystals.

Following milling and optional 60 seconds of sonication, the particle size of the finasteride particles was measured, in deionized distilled water, using a Horiba LA 910 particle size analyzer. The particle size measured is shown in Table 10 below.

TABLE 10

| | | D50 | | |
|---|---|---|---|---|
| | Mean Particle | Particle | D90 Particle | D95 Particle |
| Samples | Size (nm) | Size (nm) | Size (nm) | Size (nm) |
| Sample without | 411 | 217 | 898 | 1658 |
| sonication | | | | |
| Sample with | 211* | 167* | 261* | 511* |
| sonication | | | | |

*The particle size data marked with “*” are values that were outside of the test methods of 78-82% transmittance.

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples.

### EXAMPLE 11

The purpose of this example was to prepare a nanoparticulate formulation of finasteride.

An aqueous dispersion of 5% (w/w) finasteride, combined with 1.25% (w/w) tyloxapol, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 3500 rpms for 60 min. The sample was harvested using 21 gauge syringe after milling, demonstrating that the sample can be used in injectable formulations.

Microscopy of the milled sample, using a Lecia DM5000B and Lecia CTR 5000 light source (Laboratory Instruments and Supplies Ltd., Ashbourne Co., Meath, Ireland), showed the presence of discrete nano-particles that were susceptible to Brownian motion. There also was some localized agglomeration observed.

Following milling and optional 60 seconds of sonication, the particle size of the finasteride particles was measured, in deionized distilled water, using a Horiba LA 910 particle size analyzer. The particle size measured is shown in Table 11 below.

TABLE 11

| | | D50 | | |
|---|---|---|---|---|
| | Mean Particle | Particle | D90 Particle | D95 Particle |
| Samples | Size (nm) | Size (nm) | Size (nm) | Size (nm) |
| Sample without | 396 | 374 | 579 | 661 |
| sonication | | | | |
| Sample with | 376 | 359 | 541 | 609 |
| sonication | | | | |

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples.

### EXAMPLE 12

The purpose of this example was to prepare a nanoparticulate formulation of finasteride.

An aqueous dispersion of 5% (w/w) finasteride, combined with 1.25% (w/w) Plasdone K29/32 (Povidone K29/32) and 0.05% (w/w) lauryl sulfate (sodium lauryl sulfate), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). Sample 1 was harvested after the mixture was initially milled at a speed of 3500 rpms for 90 min. Sample 2 was harvested after the same mixture was milled for an additional 60 min at the same speed. Subsequently, the same mixture was further milled at a speed of 4500 rpm for 45 min before sample 3 was harvested. The samples were harvested using 21 gauge syringe after milling, demonstrating that the samples can be used in injectable formulations.

Microscopy of sample 3, using a Lecia DM5000B and Lecia CTR 5000 light source (Laboratory Instruments and Supplies Ltd., Ashbourne Co., Meath, Ireland), showed well dispersed nano-particles with clear evidence of Brownian motion, although there was a lot of larger drug crystals, which confirmed the distribution observation of the particle size analysis. These larger crystals appeared to be “unmilled” material. There was no sign of flocculation.

Following milling and optional 60 seconds sonication, the particle size of the finasteride particles in all three samples was measured, in deionized distilled water, using a Horiba LA 910 particle size analyzer. The particle size measured is shown in Table 12 below.

TABLE 12

| | | D50 | | D95 |
|---|---|---|---|---|
| | Mean Particle | Particle | D90 Particle | Particle |
| Samples | Size (nm) | Size (nm) | Size (nm) | Size (nm) |
| Sample 1 without | 1768 | 1423 | 3640 | 4598 |
| sonication | | | | |
| Sample 1 with | 1731 | 1403 | 3539 | 4464 |
| sonication | | | | |
| Sample 2 without | 1646 | 1436 | 3120 | 3844 |
| sonication | | | | |
| Sample 2 with | 1608 | 1405 | 3028 | 3748 |
| sonication | | | | |
| Sample 3 without | 1049 | 933 | 1840 | 2218 |
| sonication | | | | |
| Sample 3 with | 1026 | 924 | 1782 | 2136 |
| sonication | | | | |

The results demonstrate the successful preparation of stable, nanoparticulate compositions of finesteride, as the D50 particle sizes of the compositions produced were less than about 2000 nm. Moreover, the particle size measurements did not change significantly following sonication, demonstrating that aggregates of finesteride were not present in the samples. Additionally, the particle size of all three samples did not vary significantly, demonstrating that the milling time period used for the first sample was sufficient to generate a successful preparation.

It will be apparent to those skilled in the art that various modifications and variations can be made in the compositions, methods, and uses of the present invention without departing from the spirit or scope of the invention. Thus, it is intended that the present invention cover the modifications and variations of this invention provided they come within the scope of the appended claims and their equivalents.

We claim:

1. A pharmaceutical composition comprising:
(a) particles of dutasteride, tamsulosin hydrochloride, or a combination thereof having an effective average particle size of less than about 2000 nm; and
(b) at least one surface stabilizer.

2. The composition of claim 1, further comprising nanoparticulate finesteride having an effective average particle size of less than about 2000 nm in combination with at least one surface stabilizer, which can be either the same as or different from the surface stabilizer of claim 1.

3. The composition of claim 1, wherein the effective average particle size of the dutasteride or tamsulosin hydrochloride particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 mm, less than about 350 nm, less than about 300 nm, less than about 250 nm, less than about 200 nm, less than about 150 nm, less than about 100 nm, less than about 75 nm, and less than about 50 nm.

4. The composition of claim 1, wherein the composition is formulated:
(a) for administration selected from the group consisting of oral, pulmonary, rectal, opthalmic, colonic, parenteral, intracisternal, intravaginal, intraperitoneal, local, buccal, nasal, and topical administration;
(b) into a dosage form selected from the group consisting of liquid dispersions, solid dispersions, liquid-filled capsule, gels, aerosols, ointments, creams, lyophilized formulations, tablets, capsules, multi-particulate filled capsule, tablet composed of multi-particulates, compressed tablet, and a capsule filled with enteric-coated beads of a docetaxel or analogue thereof,
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c).

5. The composition of claim 4, wherein the composition is an injectable formulation

6. The composition of claim 1, wherein:
(a) the surface stabilizer is present in an amount selected from the group consisting of about 0.5% to about 99.999%, about 5.0% to about 99.9%, and about 10% to about 99.5%, by weight, based on the total combined dry weight of the dutasteride or tamsulosin hydrochloride and at least one surface stabilizer, not including other excipients;
(b) the docetaxel or analogue thereof is present in an amount selected from the group consisting of about 99.5% to about 0.001%, about 95% to about 0.1%, and about 90% to about 0.5%, by weight, based on the total combined weight of the dutasteride or tamsulosin hydrochloride and at least one surface stabilizer, not including other excipients; or
(c) a combination of (a) and (b).

7. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

8. The composition of claim 1, wherein the at least one surface stabilizer is selected from the group consisting of cetyl pyridinium chloride, albumin, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

9. The composition of claim 1, wherein the surface stabilizer is a povidone polymer.

10. The composition of claim 9, wherein the povidone polymer has a molecular weight of less than about 40,000 daltons.

11. The composition of claim 1, additionally comprising one or more non-dutasteride and non-tamsulsin hydrochloride active agents useful in treating benign prostatic hyperplasia or alopecia.

12. A injectable depot pharmaceutical composition comprising:
(a) particles of finesteride, dutasteride, tamsulosin hydrochloride, or a combination thereof having an effective average particle size of less than about 2000 nm; and
(b) at least one surface stabilizer,
wherein the injectable depot composition provides for release of finesteride, dutasteride, tamsulosin hydrochloride, or a combination thereof over an extended period of time.

13. The composition of claim 12, wherein the finesteride, dutasteride, tamsulosin hydrochloride, or a combination thereof is released over a period of time selected from the group consisting of up to about 1 week, up to about 2 weeks, up to about 3 weeks, up to about 4 weeks, up to about 5 weeks, up to about 1 month, up to about 2 months, up to about 3 months, up to about 4 months, up to about 5 months, and up to about 6 months.

14. The composition of claim 12, wherein the effective average particle size of the finesteride, dutasteride, or tamsulosin hydrochloride particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

15. The composition of claim 12, wherein:
(a) the surface stabilizer is present in an amount selected from the group consisting of about 0.5% to about 99.999%, about 5.0% to about 99.9%, and about 10% to about 99.5%, by weight, based on the total combined dry weight of the finasteride, dutasteride, or tamsulosin hydrochloride and at least one surface stabilizer, not including other excipients;
(b) the docetaxel or analogue thereof is present in an amount selected from the group consisting of about 99.5% to about 0.001%, about 95% to about 0.1%, and about 90% to about 0.5%, by weight, based on the total combined weight of the finasteride, dutasteride, or tamsulosin hydrochloride and at least one surface stabilizer, not including other excipients; or
(c) a combination of (a) and (b).

16. The composition of claim 12, wherein the surface stabilizer is selected from the group consisting of an anionic surface stabilizer, a cationic surface stabilizer, a zwitterionic surface stabilizer, a non-ionic surface stabilizer, and an ionic surface stabilizer.

17. The composition of claim 12, wherein the at least one surface stabilizer is selected from the group consisting of cetyl pyridinium chloride, albumin, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₉)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₂₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUA™, alkyl pyridinium salts; amines, amine salts, amine oxides, imide azolinium salts, protonated quaternary acrylamides, methylated quaternary polymers, and cationic guar.

18. The composition of claim 12, wherein the surface stabilizer is a povidone polymer.

19. The composition of claim 18, wherein the povidone polymer has a molecular weight of less than about 40,000 daltons.

20. The composition of claim 1, additionally comprising one or more non-dutasteride, non-finasteride, or non-tamsulsin hydrochloride active agents useful in treating benign prostatic hyperplasia or alopecia.

21. A method of treating benign prostatic hyperplasia comprising administering to a mammal an effective amount of a composition comprising:
(a) particles of finesteride, dutasteride, tamsulosin hydrochloride, or a combination thereof having an effective average particle size of less than about 2000 nm; and
(b) at least one surface stabilizer,

22. The method of claim 21, wherein the composition is formulated into an injectable depot dosage form that provides for release of the finesteride, dutasteride, tamsulosin hydrochloride, or a combination thereof over an extended period of time.

23. The method of claim 21, wherein the finesteride, dutasteride, tamsulosin hydrochloride, or a combination thereof is released over a period of time selected from the group consisting of up to about 1 week, up to about 2 weeks, up to about 3 weeks, up to about 4 weeks, up to about 5 weeks, up to about 1 month, up to about 2 months, up to about 3 months, up to about 4 months, up to about 5 months, and up to about 6 months.
